(19) United States
(12) Patent Application Publication
(10) Pub. No.: US 2006/0206050 A1
(43) Pub. Date: Sep. 14, 2006

(54) Phacoemulsification tip

(75) Inventor: James Y. Chon, Irvine, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/074,506

(22) Filed: Mar. 8, 2005

(51) Int. Cl. A61B 17/20

(52) U.S. Cl. 604/22; 604/264

(57) ABSTRACT

A phacoemulsification tip having a plurality of aspiration holes in the distal tip. By providing a plurality of aspiration pathways, the chances that the distal tip can become fully occluded are greatly reduced. Further, such a construction increases the efficiency of the tip by providing a larger contact area between the tip and the lens material being removed.

### BACKGROUND OF THE INVENTION

This invention relates generally to the field of phacoemulsification and more particularly to torsional phacoemulsification cutting tips.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an IOL.

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquefies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

One possible complication associated with cataract surgery is anterior chamber collapse following an occlusion break. Occlusion of the phacoemulsification tip can occur when a piece of lens material fully covers the distal aspiration port. When an occlusions occurs, vacuum can build in the system aspiration line so that when the occlusion eventually breaks, a sudden surge occurs, drawing fluid and lens material out of the eye and into tip aspiration port. When fluid is draw out of the eye faster than it can be replaced, the eye can soften and collapse.

One phacoemulsification tip that has gained widespread acceptance has a belled or flared distal end. Such a tip is described in U.S. Pat. No. 4,816,018 (Parisi). Such a design allows for larger lens material purchase as well as increased holding force when vacuum is applied to the tip while maintaining a smaller bore in the shaft of the tip. This combination of features increases anterior chamber stability, by reducing sudden outflow from the anterior chamber when the distal end becomes occluded and this occlusion breaks. However, occlusions can still occur.

Therefore, a need continues to exist for a phacoemulsification tip that is less prone to occlusion.

### BRIEF SUMMARY OF THE INVENTION

The present invention improves upon the prior art by providing a phacoemulsification tip having a plurality of aspiration holes in the distal tip. By providing a plurality of aspiration pathways, the chances that the distal tip can become fully occluded are greatly reduced. Further, such a construction increases the efficiency of the tip by providing a larger contact area between the tip and the lens material being removed.

Accordingly, one objective of the present invention is to provide a phacoemulsification cutting tip having increased efficiency.

Another objective of the present invention is to provide a phacoemulsification cutting tip having a plurality of aspiration ports on the distal tip.

These and other advantages and objectives of the present invention will become apparent from the detailed description and claims that follow.

### BRIEF DESCRIPTION OF THE DRAWINGS

FIG. 1 is an enlarged perspective view of a typical prior art phacoemulsification tip.

FIG. 2 is an enlarged perspective view of the phacoemulsification tip of the present invention.

FIG. 3 is an enlarged perspective view of a distal tip of the present invention formed as a separate piece from the phacoemulsification tip illustrated in FIG. 2.

### DETAILED DESCRIPTION OF THE INVENTION

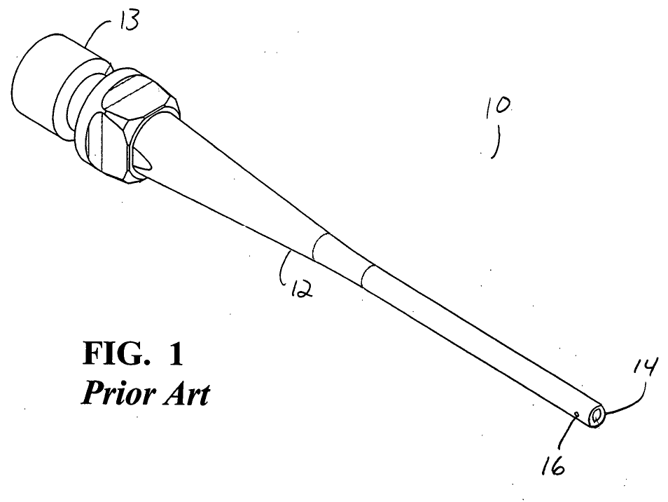

As best seen in FIG. 1, prior art phacoemulsification tip 10 contains shaft 12 extending from hub 13. Shaft 12 is straight all the way to distal tip 14. Distal tip 14 may be angled or bent relative to the centerline of shaft 12. Shaft 12 may contain aspiration bypass hole 16.

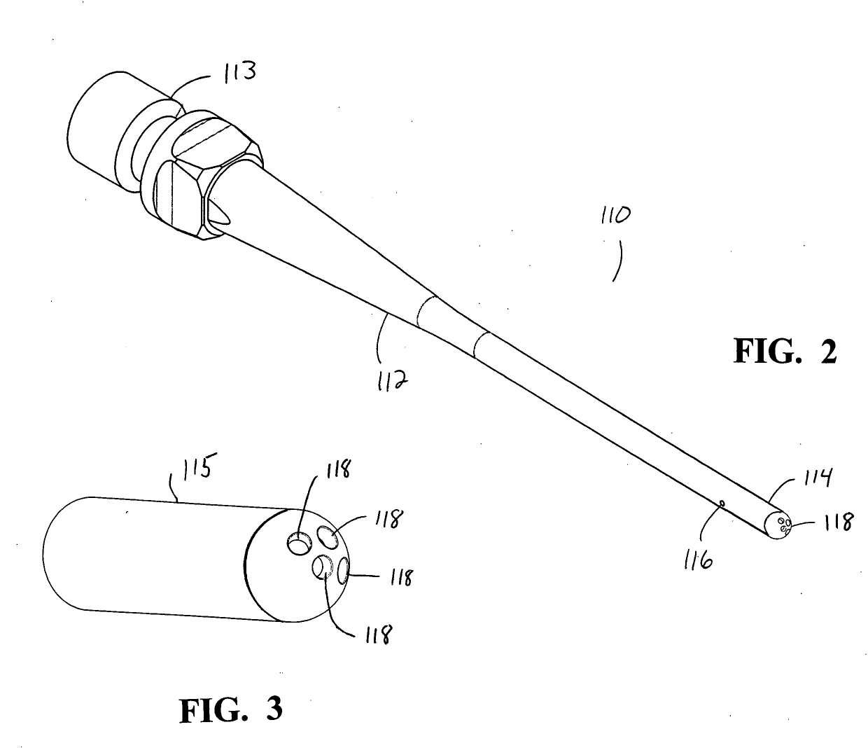

As best seen in FIG. 2, phacoemulsification tip 110 contains shaft 112 extending from hub 113. Shaft 112 is straight all the way to distal tip 114. Distal tip 114 may be angled or bent relative to the centerline of shaft 112. Shaft 112 may contain aspiration bypass hole 116.

As best seen in FIGS. 2 and 3, distal tip 114 is generally rounded and enclosed but contains a plurality of aspiration ports 118. The blunt, rounded shape of distal tip 114 increases the contact area between tip 110 and the material being removed and is also less likely to cause damage to delicate structures in the eye, such as the posterior capsule. By using a plurality of ports 118, tip 110 is less likely to become occluded, thereby reduces the chances for post-occlusion surge. While FIG. 2 shows distal tip 114 integrally formed with shaft 112, one skilled in the art will recognize that distal tip 114 can be formed as a separate tip cap 115, as shown in FIG. 3, sized and shaped to fit over distal tip 14 of convention phacoemulsification tip 10.

Tip 110 is preferably made from stainless steel or titanium, but other materials may also be used. Tip 110 preferably has an overall length of between 0.50 inches and 1.50 inches, with 1.20 inches being most preferred. Tip 110 may be formed using conventional metalworking technology and preferably is electropolished to remove any burrs.

Shaft 112 is generally tubular, with an outside diameter of between 0.005 inches and 0.100 inches and an inside diameter of between 0.001 inches and 0.090 inches.

This description is given for purposes of illustration and explanation. It will be apparent to those skilled in the relevant art that changes and modifications may be made to the invention described above without departing from its scope or spirit.

I claim:

1. A phacoemulsification tip, comprising: a tubular shaft having a distal end, the distal having a plurality of aspiration ports.

2. The phacoemulsification tip of claim 1 wherein the shaft contains an aspiration bypass hole.

3. The phacoemulsification tip of claim 1 wherein the distal end is generally rounded.
